(19) United States
(12) Patent Application Publication
(10) Pub. No.: US 2016/0177357 A1
(43) Pub. Date: Jun. 23, 2016

(54) METHOD FOR PRETREATING CELLULOSE-CONTAINING BIOMASS, METHOD FOR PRODUCING BIOMASS COMPOSITION FOR SACCHARIFICATION USE, AND METHOD FOR PRODUCING SUGAR

(71) Applicant: SHOWA DENKO K.K., Tokyo (JP)

(72) Inventors: Ichiro FUJITA, Tokyo (JP); Shoichiro WAKABAYASHI, Tokyo (JP); Shinji YAMAKI, Tokyo (JP); Tadashi YONEDA, Tokyo (JP)

(73) Assignee: SHOWA DENKO K.K., Tokyo (JP)

(21) Appl. No.: 14/893,114

(22) PCT Filed: Mar. 28, 2014
(86) PCT No.: PCT/JP2014/059290

§ 371 Date: Nov. 23, 2015

(30) Foreign Application Priority Data
May 30, 2013 (JP) 2013-114526

(51) Int. Cl. C12P 19/14; C08B 1/00; C12P 19/02

(52) U.S. Cl. CPC C12P 19/14; C12P 19/02; C08B 1/00; C12P 2201/00

(57) ABSTRACT

A pretreatment method for producing sugar from a cellulose-containing biomass, which includes subjecting the cellulose-containing biomass to hydrothermal treatment while fluidizing the cellulose-containing biomass; a production method of a biomass composition for saccharification use, including the pretreatment method; and a method for producing sugar, which includes hydrolyzing the biomass composition for saccharification use obtained by the above-mentioned production method.

### TECHNICAL FIELD

The present invention relates to production of a sugar through hydrolysis of cellulose-containing biomass. Particularly, the present invention relates to a method for pretreating cellulose-containing biomass capable of obtaining a cellulose-containing composition, which can be saccharified into glucose at a high level; and a method for producing a sugar.

### BACKGROUND ART

As part of measures against global warming, there have been wide investigations on production of various chemical products including ethanol through effective utilization of cellulose-containing biomass. Examples of the cellulose-containing biomass include hard biomass such as cedar or cypress, and soft biomass such as rice straw, wheat straw, corncobs, cassava, bagasse, or sugar cane leaves. The biomass may contain hemicellulose, lignin, and the like, and hence it is difficult to directly saccharify the biomass. Therefore, there have been proposals to enhance its saccharification performance through various pretreatments.

As classical pretreatment methods, acid treatment, alkali treatment, hydrothermal treatment, and the like have been proposed. The acid treatment is a technology capable of effectively removing hemicellulose as an impurity, but has a problem in that its industrial practice entails high cost owing to corrosion of a device due to an acid and necessity of neutralization of the used acid in a subsequent step. The alkali treatment is a technology capable of effectively removing lignin as an impurity, but has a problem in that its industrial practice entails high cost owing to a large cellulose loss and decrease in production per material unit. On the other hand, regarding the hydrothermal treatment, which involves heating the biomass together with water in a sealed container, there has been a proposal to enhance its pretreatment effect by combination use with physical pulverization treatment, because the hydrothermal treatment does not use a chemical such as an acid or an alkali and hence has a low treatment effect (JP 2006-136263 A; Patent Document 1). However, industrially useful treatment conditions are not explicitly disclosed.

Other than the above-mentioned methods, there have been proposed water vapor blasting, ammonia blasting, ozone oxidation, white-rot fungus treatment, microwave irradiation, electron beam irradiation, and γ-ray irradiation (Journal of The Japan Wood Research Society, 53, 1 to 13 (2007); Non Patent Document 1). However, those methods are treatment methods involving high facility cost and high chemical cost, and are insufficient for industrial practice from the viewpoint of cost-effectiveness.

For these reasons, there has been demand for establishment of a pretreatment method for cellulose-containing biomass capable of obtaining a biomass composition for saccharification that exhibits high saccharification performance and is industrially useful, a method for producing a biomass composition for saccharification by conducting the pretreatment method, and a method for producing a sugar through hydrolysis of the biomass composition for saccharification.

### PRIOR ART

### Patent Document

[Patent Document 1] JP 2006-136263 A

### Non-Patent Document

[Non-patent Document 1] Journal of The Japan Wood Research Society, 53, 1 to 13 (2007)

### DISCLOSURE OF THE INVENTION

### Problems to be Solved by the Invention

An object of the present invention is to provide a method for pretreating cellulose-containing biomass capable of obtaining a cellulose-containing composition, which can be saccharified into glucose at a high level; a method for producing a biomass composition for saccharification by conducting the pretreatment method; and a method for producing a sugar through hydrolysis of the cellulose-containing composition for saccharification.

### Means to Solve the Problem

As a result of diligent investigations aimed at achieving the above-mentioned object, the inventors of the present invention have found that, in pretreatment of cellulose-containing biomass, it is possible to obtain a cellulose-containing composition which can be saccharified into glucose at a high level by subjecting the cellulose-containing biomass to hydrothermal treatment while fluidizing the cellulose-containing biomass. Thus, the present invention has been completed.

That is, the present invention relates to the following method for pretreating cellulose-containing biomass, a method for producing a cellulose-containing composition for saccharification, and a method for producing a sugar.

[1] A pretreatment method for cellulose-containing biomass, for enhancing saccharification performance in a hydrolysis reaction, comprising subjecting the pulverized cellulose-containing biomass to hydrothermal treatment while fluidizing the cellulose-containing biomass.
[2] The pretreatment method for cellulose-containing biomass, for enhancing saccharification performance in a hydrolysis reaction according to [1] above, comprising subjecting the pulverized cellulose-containing biomass to hydrothermal treatment while fluidizing the cellulose-containing biomass by setting Reynolds number to 0.5 or more.
[3] The pretreatment method according to [1] or [2] above, wherein the pulverized cellulose-containing biomass is fluidized by stirring.
[4] The pretreatment method according to any one of [1] to [3] above, wherein the cellulose-containing biomass is soft biomass.
[5] The pretreatment method according to any one of [1] to [4] above, wherein the cellulose-containing biomass is pulverized using a screen having a mesh of from 1 to 30 mmφ.
[6] The pretreatment method according to any one of [1] to [5] above, in which the hydrothermal treatment includes heating a mixture of the pulverized cellulose-containing biomass and water at a temperature of from 180 to 250° C. for from 1 to 100 minutes.
[7] The pretreatment method according to any one of [1] to [6] above, in which a mass ratio between the cellulose-containing biomass in terms of dry mass and water in the hydrothermal treatment is from 1:4 to 1:97.
[8] The pretreatment method according to any one of [1] to [7] above, in which the hydrothermal treatment is performed by using water having a pH of from 5.8 to 8.6.
[9] A method for producing a biomass composition for saccharification, the method comprising conducting the pretreatment method described in any one of [1] to [8] above.
[10] A method for producing a sugar, comprising hydrolyzing a biomass composition for saccharification obtained by the production method described in [9] above.

### Effects of Invention

According to the pretreatment method for cellulose-containing biomass of the present invention, comprising subjecting pulverized cellulose-containing biomass to hydrothermal treatment while fluidizing the cellulose-containing biomass, a cellulose-containing composition for saccharification useful as a raw material for producing a sugar through a hydrolysis reaction can be obtained. Thus, sugar can be efficiently produced from cellulose-containing biomass.

### BRIEF DESCRIPTION OF DRAWINGS

FIG. 1 shows a temperature profile of the hydrothermal treatment in Examples 1 to 5 and Comparative Example 1.

FIG. 2 is a graph showing the results (relationship between the Reynolds number at room temperature before starting the hydrothermal treatment and the saccharification rate) in Examples 1 to 5 and Comparative Example 1.

### MODE FOR CARRYING OUT THE INVENTION

Hereinafter, the present invention is described in details.

### [Cellulose-Containing Biomass]

The biomass in the present invention means a biopolymer (nucleic acid, protein, or polysaccharide) or an industrial resource derived from such constituent component, other than exhaustible resources (fossil fuel such as petroleum, coal, or natural gas). Therefore, examples of the cellulose-containing biomass include hard biomass such as wood, and soft biomass such as rice straw, wheat straw, corncobs, cassava, bagasse, or sugar cane leaves. Soft biomass is preferred in consideration of the ease of the pretreatment, and further, bagasse and sugar cane leaves are particularly preferred in consideration of their global storage potential and collection cost.

[Pulverization (Adjustment of Grain Diameter)]

In the present invention, it is desirable to adjust the grain diameter by pulverizing the cellulose-containing biomass as a raw material before thermal treatment. The pulverization means is not particularly limited as long as the means has a function to enable fine pulverization of a solid substance. For example, the mode of the apparatus may be a dry mode or a wet mode. In addition, the pulverization system of the apparatus may be a batch system or a continuous system. Further, as a pulverization apparatus, an apparatus using any kind of pulverizing force provided by impact, compression, shearing, friction and the like can be used.

As a specific apparatus which can be used for the pulverization treatment, preliminary pulverization treatment may be performed before the fine pulverization with, for example: a coarse crusher such as a shredder, a jaw crusher, a gyratory crusher, a cutter mill, a cone crusher, a hammer crusher, a roll crusher or a roll mill; or a medium crusher such as a stamp mill, an edge runner, a cutting/shearing mill, a rod mill, an autogenous mill or a roller mill. From the viewpoint of the throughput and the range of pulverization, a cutter mill is preferred. The time for treating the raw material is not particularly limited as long as the raw material can be homogeneously and finely pulverized by the treatment.

With respect to the grain size after the adjustment, in the case where the screen diameter is too large in the pulverization, the grain size of the cellulose-containing biomass becomes large, and hence a pretreatment effect to be obtained through subsequent steps lowers and the production cost of a sugar becomes high. In the case where the screen diameter is too small in the pulverization, pulverization cost becomes high. Therefore, the grain of the biomass preferably has such a size that allows the grain to pass through a screen (sieve) having a screen diameter of from 1 to 30 mmφ. The screen diameter falls within a range of more preferably from 5 to 30 mmφ, most preferably from 10 to 30 mmφ. In the case of pulverizing the cellulose-containing biomass without using the screen, it is preferred that the biomass be pulverized to the size corresponding to that of a pulverized product obtained by using the screen.

[Adjustment of Water Content]

In the present invention, water content of the biomass is adjusted before performing the hydrothermal treatment.

Examples of the method for adjusting the water content includes adding water, dehydrating, or drying in accordance with the water content of the raw material before the adjustment.

If the amount of water is too large with respect to the cellulose-containing biomass in terms of dry mass, it increases the cost due to the increase in the scale of a pretreatment device. If the amount of water is too small with respect to the cellulose-containing biomass in terms of dry mass, the pretreatment effect lowers, and the production cost of the sugar becomes high. Therefore, the mass ratio between the cellulose-containing biomass in terms of dry mass and water in the hydrothermal treatment is preferably from 1:4 to 1:97. The mass ratio is more preferably from 1:6 to 1:20, most preferably from 1:8 to 1:13.

In the present invention, it is desirable to adjust the chlorine ion concentration prior to the process of adjusting the water content by washing the cellulose-containing biomass with water. After subjecting the cellulose-containing biomass to the hydrothermal treatment with no addition of chemical agents, the pH of the biomass generally shifts to a weak acid range of 3 to 5. When a commonly-used material of the device such as stainless steel and super stainless steel is exposed to high temperature for a long period of time under acidic condition in coexistence with chloric ions, it is likely to give rise to stress corrosion cracks. On the other hand, when the chlorine ion concentration is too low, it increases the water usage for washing and the treatment time, resulting in high cost for producing sugar. Therefore, the chlorine concentration in the biomass slurry after the adjustment of water content is controlled preferably to 0.1 to 300 ppm, more preferably 1 to 200 ppm, still more preferably 5 to 100 ppm.

[Hydrothermal Treatment]

In the present invention, the slurry of the cellulose-containing biomass is subjected to thermal treatment while fluidizing the slurry. Furthermore, the slurry is subjected to thermal treatment while fluidizing the slurry by setting Reynolds number (Re), which is a dimensionless number represented by the below equation and defined as the ratio of the fictitious force and the viscous force in fluid dynamics, to preferably 0.5 or more.

$$Re = \frac{ρ \times n \times {d}^{2}}{μ}$$

In the formula, ρ represents the density of the biomass slurry (kg/m³), n represents the rotation rate (rps), d represents the blade span (m), and μ represents the viscosity (Pa·S).

When the maximum treatment temperature in the hydrothermal treatment heating temperature is too high, energy cost becomes rather high, and in addition, decomposition of cellulose or excessive decomposition of impurities proceeds. When the maximum treatment temperature is too low, the pretreatment effect lowers, and the production cost of the sugar becomes high. Accordingly, the temperature is set to preferably 180 to 250° C., more preferably 180 to 230° C., most preferably 180 to 220° C.

In the hydrothermal treatment, the retention time period at a temperature of 95% or higher of the maximum treatment temperature is preferably from 1 to 100 minutes. The retention time period is more preferably from 3 to 75 minutes, most preferably from 6 to 50 minutes. If the retention time period is too long, the productivity in the pretreatment steps lowers, and hence the production cost of the sugar becomes high. If the retention time period is too short, the pretreatment effect lowers, and hence the production cost of the sugar becomes high. It should be noted that the preferred range of the heating time period varies within the above-mentioned range depending on the heating temperature to be adopted.

An acid or an alkali may be added as an additive to water in the hydrothermal treatment of the present invention. However, the use of an additive entails chemical cost, and cost for detoxification by neutralization or the like in a subsequent step as well. Therefore, it is preferred from an industrial viewpoint to use only water, which is generally available.

[Separation by Dehydration]

The process of separation by dehydration aims to increase the solid content of the product subjected to thermal treatment and to collect the soluble component contained in the product to thereby reduce the concentration of the soluble component.

Considering the use of the saccharified solution produced by saccharification of the cellulose-containing biomass as the fermentation feedstock, if sugar is sterilized at the same time with protein as the main component of the medium, it causes Maillard reaction and may inhibit the fermentation or increase the separation and refinement load in the fermentation due to the pigmentation. Hence, sugar is generally sterilized separately to be supplied. Accordingly, a higher concentration of sugar is required compared to the case where the sugar and protein are sterilized at one time. It is also necessary to further increase the concentration of sugar as a raw material in order to improve the sugar concentration accumulated in the fermentation product. If the sugar concentration in the saccharified solution is too low, it requires a condensation process and increase the production cost, which is not desirable. Thus, the reason for increasing the solid content concentration by dehydration is that it is necessary to increase the concentration of the substrate in the saccharification reaction: i.e. to increase the solid content concentration in order to obtain a saccharification solution having a high sugar concentration that is of practical value.

On the other hand, in view of the soluble components in the product subjected to hydrothermal treatment, soluble components in the hydrothermal reaction with no addition of chemical agents include xylose, arabinose, galactose, xyloligosaccharide as a decomposed product of hemicellulose, and furfural, levoglucosan and 5-hydroxymethylfurfural as an overdecomposition product. Therefore, collecting the soluble components by dehydration is useful from the viewpoint of highly utilizing hemicellulose and from the viewpoint of suppressing the inhibition of fermentation and saccharification due to an overdecomposition product.

As a method for dehydration, solid-liquid separation can be conducted by an apparatus such as a filter press, Oliver filter, centrifugal filter and centrifugal separator.

If the amount of water is too small with respect to the product subjected to hydrothermal treatment after dehydration in terms of dry mass, it results in a high production cost due to the increase in the scale of a pretreatment device and treatment time. Thus, the amount of water is preferably 0.4 to 6 times, more preferably 1 to 4 times, most preferably 2 to 2.5 times amount of the product subjected to hydrothermal treatment in terms of dry mass.

In the present invention, a process of finely pulverizing the product subjected to hydrothermal treatment by a dry-method refiner or a disk mill may be provided between the process of hydrothermal treatment and the process of separation by dehydration. The pulverization step is performed a plurality of times until a biomass composition is pulverized to have an average particle size of 300 μm or less in a dry state.

If the number of times of pulverization is too small, the pretreatment effect lowers, and the production cost of the sugar becomes high. If the number of times of pulverization is too large, the pulverization cost becomes high. Hence, the number of times of pulverization is preferably from 4 to 50, more preferably from 6 to 30.

By the pretreatment method comprising the above-mentioned process of preparing a raw material, process of hydrothermal treatment and process of separation by dehydration, a cellulose-containing composition, which can be saccharified into glucose at a high level, can be obtained.

As a method of hydrolyzing and saccharifying the obtained biomass composition for saccharification, there is given a method of hydrolyzing cellulose by using a solid acid catalyst or a mineral acid catalyst such as sulfuric acid or a method of hydrolyzing cellulose by using an enzyme. The hydrolysis method using an enzyme is industrially advantageous because impurities are generated in small amounts and the obtained sugar has a high utility value.

The hydrolysis of cellulose with an enzyme is performed by, for example, allowing a generally known cellulase to act on the biomass composition for saccharification of the present invention. While the properties of the cellulase slightly vary depending on the kind of the cellulase, an optimum pH falls within a range of from 3.5 to 5.5 and an optimum temperature falls within a range of from 35 to 55° C. Therefore, cellulose is hydrolyzed by treating the biomass composition at a temperature of from 35 to 55° C. for a predetermined time period after a buffer solution having a pH of from 3.5 to 5.5 is added thereto. Thus, the sugar can be produced.

### EXAMPLES

Hereinafter, the present invention is described by way of Example and Comparative Example. However, the present invention is not limited to the descriptions of Example and Comparative Example.

[Analysis Method for Contents of Main Components in Biomass]

The total content of cellulose content, hemicellulose content and lignin content in the biomass was determined by the analysis method (Technical Report NREL/TP-510-42618) of NREL (U.S. National Renewable Energy Laboratory).

[Measurement Method of the Density of Biomass Slurry]

The density (kg/m³) of the biomass slurry before hydrothermal treatment was determined as follows. 100 g of slurry was divided after fully mixing and placed in a measuring cylinder having a full capacity of 200 ml at 25° C. After removing bubbles in the slurry sufficiently, the volume of the slurry was measured. The density was calculated from the measured values of the mass and volume.

[Measurement Method of the Viscosity of Biomass Slurry]

With respect to the viscosity (Pa·s) of the biomass slurry before hydrothermal treatment, fully mixed slurry was divided and measured at 25° C. using the BLII-type viscosity meter manufactured by Toki Sangyo Co., Ltd. provided with a No. 4 rotor at a rotation rate of 6 rpm.

[High-Performance Liquid Chromatography Analysis Method and Calculation Method for Content Rate of Cellulose]

A guard column (KS-G manufactured by Showa Denko K.K.) and a separation column (KS-802 manufactured by Showa Denko K.K.) were connected to each other, and the column temperature was set to 75° C. Pure water was supplied as an eluting solution at a rate of 0.5 ml/min, and a separated component was subjected to quantitative determination with a differential refractive index detector. Thus, the concentration of glucose was determined, and the content rate of cellulose was calculated based on the following equation.

Content rate of cellulose (mass %)=mass of filtrate (g)×concentration of glucose (mass %×0.01)×0.9/mass of weighed biomass (g) [Equation 1]

The numerical value “0.9” in the equation is a coefficient for correcting changes in molecular weight caused by hydrolysis of cellulose.

[Measurement of Saccharification Performance with Enzyme]

### Preparation of Acid Buffer Solution:

30 g of acetic acid was put in a 100-ml measuring flask, and diluted with pure water to give a 5 M acetic acid aqueous solution. 41 g of sodium acetate was put in a 100-ml measuring flask, and diluted with pure water to give a 5 M sodium acetate aqueous solution. The 5 M acetic acid aqueous solution was added to the 5 M sodium acetate aqueous solution until the pH reached 5.0 to obtain an acetic acid buffer solution.

Preparation of Enzyme Solution:

1.5 g of Meicelase (trademark, cellulase manufactured by Meiji Seika Kaisha, Ltd. (current Meiji Seika Pharma Co., Ltd.)) was dissolved in 98.5 g of pure water.

As a result of the analysis method (Pure & Appl. Chem., Vol. 59, No. 2, pp. 257-268, 1987) according to IUPAC (International Union of Pure and Applied Chemistry), the FPU activity (Filter Paper Assay for Saccharifying Cellulase) of the enzyme solution was 6 FPU/g.

Saccharification Reaction:

A rotor was put in a 50-ml glass vessel with a cover, and a composition subjected to pretreatment was weighed so as to contain 0.5 g of cellulose and was put in the vessel. Then, 0.6 g of the acetic acid buffer solution and 1.03 g of the enzyme solution were added thereto, and further pure water was added thereto to give a total of 10 g. The resultant was subjected to a saccharification reaction with the enzyme in a constant temperature bath at 40° C. for 48 hours while being stirred. The resultant saccharified solution was subjected to quantitative determination for glucose by high-performance liquid chromatography analysis. Thus, a saccharification rate and a sugar availability were determined by the following formula.

Saccharification rate=glucose concentration (%) of the reaction solution×0.9/cellulose concentration (%) of the reaction solution

[Preparation of the Raw Material Bagasse]

Bagasse was used as the cellulose-containing biomass serving as a raw material.

Air-dried bagasse was pulverized with a cutter mill (manufactured by Masuko Sangyo Co., Ltd.; MKCM-3) having a 10 mmφ screen and a cutter mill (manufactured by Masuko Sangyo Co., Ltd.; MKCM-3) having a 3 mmφ screen to obtain a bagasse referred to as “10 mm bagasse” (water content: 10.5%) and “3 mm bagasse” (water content 10.3%), respectively. The obtained “3 mm bagasse” was pulverized with a blender (manufactured by Hamilton Beach Brands, Inc.; HBF500S) and allowed to pass through a sieve having a mesh of 0.5 mm to obtain a bagasse referred to as “0.5 mm bagasse” (water content: 10.3%). The thus obtained bagasse was used as the adjusted bagasse.

### Comparative Example 1

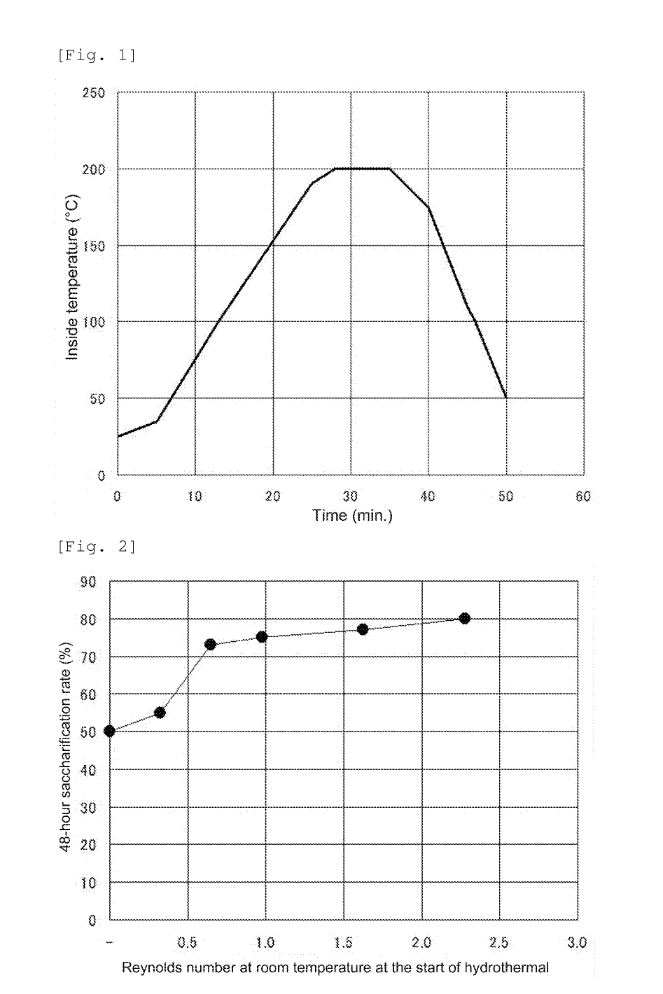

13.44 g of the 0.5 mm bagasse and 106.56 g of pure water were put in a 300-ml autoclave having no agitator (high-pressure micro reactor MMJ-300 manufactured by Om Lab-Tech Co., Ltd.) to be sealed and hydrothermal treatment was conducted by controlling the heating and cooling to comply with the temperature profile shown in FIG. 1. The resultant slurry was subjected to centrifugal filtration with a centrifugal filtration device (manufactured by Kokusan Co., Ltd.; H-122; cotton filter cloth) at 3,000 rpm, to obtain a water-containing solid content.

The obtained water-containing solid content was evaluated for a saccharification rate by the above-mentioned method.

### Examples 1 to 5

447 g of “0.5 mm bagasse” was put in a 10-1 autoclave (desktop reactor OML-10 manufactured by Om Lab-Tech Co., Ltd.).

Further, 3,953 g of pure water was put therein, and the autoclave was sealed. While stirring at a rotation rate of 100 rpm in Example 1, 200 rpm in Example 2, 300 rpm in Example 3, 400 rpm in Example 4 and 500 rpm in Example 5, hydrothermal treatment was conducted by controlling the heating and cooling to comply with the temperature profile shown in FIG. 1.

The resultant slurry was subjected to centrifugal filtration with a centrifugal filtration device (manufactured by Kokusan Co., Ltd.; H-122; cotton filter cloth) at 3,000 rpm, to obtain a water-containing solid content. The obtained water-containing solid content was evaluated for a saccharification rate by the above-mentioned method.

The results of Examples 1 to 5 and Comparative Example 1 are shown in Table 1.

TABLE 1

| | | | | | | Reynolds | |
|---|---|---|---|---|---|---|---|
| | | | Rotation | | | Number | 48-hour |
| | Density (ρ) | Rotation | rate (n) | Blade span | Viscosity (μ) | (Re) | saccharification |
| | (kg/m³) | rate (rps) | (rps) | (d) (m) | (Pa · s) | Re = ρ · n · d²/μ | rate (%) |
| Comparative | 1040 | 0 | 0 | 0 | 30 | 0.0 | 50 |
| Ex. 1 | | | | | | | |
| Example 1 | 1040 | 100 | 1.7 | 0.075 | 30 | 0.3 | 55 |
| Example 2 | 1040 | 200 | 3.3 | 0.075 | 30 | 0.7 | 73 |
| Example 3 | 1040 | 300 | 5.0 | 0.075 | 30 | 1.0 | 75 |
| Example 4 | 1040 | 500 | 8.3 | 0.075 | 30 | 1.6 | 77 |
| Example 5 | 1040 | 700 | 11.7 | 0.075 | 30 | 2.3 | 80 |

The biomass slurry before hydrothermal treatment had a density of 1,040 kg/m³ and a viscosity of 30 Pa·s in any of Examples 1 to 5 and Comparative Example 1. The saccharification rate after 48-hour saccharification reaction was 50% in Comparative Example 1, in which hydrothermal treatment was conducted without stirring and the Reynolds number at room temperature before hydrothermal treatment was 0. In contrast, the Reynolds number at room temperature and the saccharification rate were 0.3 and 55% in Example 1, 0.7 and 73% in Example 2, 1.0 and 75% in Example 3, 1.6 and 77% in Example 4 and 2.3 and 80% in Example 5, respectively. The saccharification rate was higher compared to Comparative Example in all the Examples, and the saccharification rate increased with the Reynolds number.

Furthermore, as can be seen from FIG. 2 showing the relationship between the Reynolds number and saccharification rate, the increase in the saccharification rate between Example 1 and Example 2 is particularly larger compared to the increase between other Examples. This suggests that the Reynolds number around 0.5 is the inflection point. In view of this, it can be said that it is desirable to set the Reynolds number 0.5 or higher to obtain a cellulose-containing composition having a higher saccharification rate.

As seen from the above results, it was confirmed that a cellulose-containing composition having a higher saccharification rate can be obtained when the hydrothermal treatment is conducted while fluidizing the cellulose-containing biomass.

1. A pretreatment method for cellulose-containing biomass, for enhancing saccharification performance in a hydrolysis reaction, comprising subjecting the pulverized cellulose-containing biomass to hydrothermal treatment while fluidizing the cellulose-containing biomass.

2. The pretreatment method for cellulose-containing biomass, for enhancing saccharification performance in a hydrolysis reaction according to claim 1, comprising subjecting the pulverized cellulose-containing biomass to hydrothermal treatment while fluidizing the cellulose-containing biomass by setting Reynolds number to 0.5 or more.

3. The pretreatment method according to claim 1, wherein the pulverized cellulose-containing biomass is fluidized by stirring.

4. The pretreatment method according to claim 1, wherein the cellulose-containing biomass is soft biomass.

5. The pretreatment method according to claim 1, wherein the cellulose-containing biomass is pulverized using a screen having a mesh of from 1 to 30 mmφ.

6. The pretreatment method according to claim 1, in which the hydrothermal treatment includes heating a mixture of the pulverized cellulose-containing biomass and water at a temperature of from 180 to 250° C. for from 1 to 100 minutes.

7. The pretreatment method according to claim 1, in which a mass ratio between the cellulose-containing biomass in terms of dry mass and water in the hydrothermal treatment is from 1:4 to 1:97.

8. The pretreatment method according to claim 1, in which the hydrothermal treatment is performed by using water having a pH of from 5.8 to 8.6.

9. A method for producing a biomass composition for saccharification, the method comprising conducting the pretreatment method described in claim 1.

10. A method for producing a sugar, comprising hydrolyzing a biomass composition for saccharification obtained by the production method described in claim 9.
